(19) United States
(12) Patent Application Publication
(10) Pub. No.: US 2025/0020550 A1
(43) Pub. Date: Jan. 16, 2025

(54) SAMPLE COLLECTION DEVICE AND SYSTEM

(71) Applicant: Hound Labs, Inc., Fremont, CA (US)

(72) Inventors: Robert Duck, Fremont, CA (US); Debkishore Mitra, Fremont, CA (US); Spencer Sawyer, Fremont, CA (US); Gage Fain, Fremont, CA (US); Hoang Nguyen, Fremont, CA (US); Daniel H. Friedman, Fremont, CA (US)

(21) Appl. No.: 18/614,599

(22) Filed: Mar. 22, 2024

Related U.S. Application Data
(60) Provisional application No. 63/454,050, filed on Mar. 22, 2023.

(51) Int. Cl. G01N 1/22

(52) U.S. Cl. CPC G01N 1/2202; G01N 33/4975

(57) ABSTRACT

Examples include a sample collection device that includes a housing, a reservoir structure and a sample capture interface. The housing includes an inlet to receive an air sample from a subject. The sample capture interface provided with the housing includes a sample capture medium. The sample capture interface is manipulatable to insert the sample capture medium into the reservoir structure. Further, the sample capture interface seals the sample capture medium within the reservoir structure. Upon insertion of the sample capture interface, the sample capture interface seals the sample capture medium within the reservoir structure.

### RELATED APPLICATIONS

This application claims benefit of priority to Provisional U.S. Patent Application No. 63/454,050, filed Mar. 22, 2023; the aforementioned priority application being hereby incorporated by reference in its entirety.

### BACKGROUND

With legalization of marijuana expanding and the risk of marijuana-associated impaired driving increasing, there is an increased need for portable and accurate measurement systems, methods and devices for quantifying levels of cannabinoid compounds, such as tetrahydrocannabinol (THC), that are present in a person's breath, e.g., such as during a traffic stop for suspected driving-under-the-influence. THC detection poses significant challenges since the amounts of THC that may be present in an exhaled breath are quite minute-much more so than is the case with alcohol.

### BRIEF DESCRIPTION OF DRAWINGS

FIG. 1A through FIG. 1C illustrate a sample collection device and system, according to one or more embodiments.

FIG. 2A through FIG. 2C illustrate a sample collection device, in accordance with one or more embodiments.

FIG. 2D and FIG. 2E illustrate a reservoir structure for use with an embodiment of FIG. 2A through FIG. 2C.

FIG. 3A through FIG. 3C provide an exploded view of a sample collection device, according to one or more examples.

FIG. 3D and FIG. 3E illustrates an example of cartridge for use with one or more embodiments, according to one or more examples.

FIG. 3F through FIG. 3H illustrate components of a sample collection device being manipulated to immerse the sample captured medium (with the captured sample) in a solvent for transport to a remote site.

FIG. 4A and FIG. 4B illustrate another example of a sample capture device, according to one or more embodiments.

FIG. 4C illustrates an exploded view of the sample collection device, according to one or more embodiments.

FIG. 4D illustrates a bottom view of the sample collection device, according to one or more embodiments.

FIG. 4E illustrates an example of a sample capture medium, according to one or more embodiments.

FIG. 5A and FIG. 5B illustrate another example of a sample capture device, according to one or more embodiments.

FIG. 5C illustrates a cross-sectional view of the sample capture device of FIG. 5A and FIG. 5B, along an axial axis Z (coinciding with lines A-A), according to one or more embodiments.

FIG. 5D through FIG. 5F illustrate mechanisms of a sample collection device that combine to enable a collected sample to be inserted and sealed within a reservoir structure, according to one or more embodiments.

FIG. 5G is a closeup of region C of FIG. 5F, illustrating an example mechanism for automatically sealing a sample capture medium within a reservoir structure, according to one or more embodiments.

FIG. 5H illustrates a bottom perspective view of the region C, further illustrating a mechanism for sealing a reservoir structure to prevent leakage after immersion of a sample capture medium, according to one or more embodiments.

FIG. 5I is a closeup of region D of FIG. 5F, illustrating an example of the housing segments being structured to form a lock when manipulated into a collapsed position, according to one or more embodiments.

FIG. 5J illustrates a partial perspective and cross-sectional view of a base structure and a plunger structure when the respective housing segments, are in an expanded state (e.g., before or during sample collection).

FIG. 5K illustrates another cross-sectional view of sample collection device 500 when in the extended state, according to one or more embodiments.

FIG. 5L and FIG. 5M are perspective cut-away views of FIG. 5K, showing the sample collection device with a top housing segment removed, while in a partially and fully collapsed state, respectively, according to one or more embodiments.

FIG. 5N illustrates an example of the base structure for a housing segment of a sample capture device, according to one or more embodiments.

FIG. 5O illustrates an example of sample collection device that utilizes an X-ring for a deformable layer, according to one or more embodiments.

FIG. 6 illustrates a flow path for a sample intake of a sample capture device, according to one or more embodiments.

FIG. 7A through FIG. 7D illustrate use of a sample capture device, in accordance with one or more embodiments.

FIG. 8A through FIG. 8C illustrates a sample collection device during a sample intake process, according to one or more embodiments.

### DETAILED DESCRIPTION

According to embodiments, a sample capture device includes a housing having an inlet to receive a sample, and a sample capture interface provided with the housing. In embodiments, the sample capture interface is structured to (i) retain a sample capture medium, (ii) insert the sample capture medium with a captured sample (e.g., particles in exhaled breath or ambient air) into a reservoir structure, and (iii) seal the sample capture medium within the reservoir structure. The reservoir structure can include a preservation fluid to preserve the sample. As described with examples, the reservoir structure can be shipped or otherwise transported as part of a protective housing segment, where the captured sample can be removed and evaluated.

In embodiments, the sample capture device can be structured to receive and seal a sample within a preservation solution. The device can then be transported to a laboratory where the sample is analyzed for one or more target substances. In examples, the target substance includes tetrahydrocannabinol (THC) or other markers such as such as cannabinol (CBN), cannabidiol (CBD), carboxy THC or 11-nor-9-carboxy-49-tetrahydrocannabinol (THC-COOH), 11-hydroxy-49-tetrahydrocannabinol (11-hydroxy THC), 9-carboxy THC or 49-tetrahydrocannabinolic acid (THC-9-COOH), tetrahydrocannabinolic acid (THCA, THC-2-COOH), and similar compounds. However, in variations, the sample collection device 100 can be utilized to detect numerous types of analytes, including markers of diseases such as cancer, as well as indicators of other types of substances of interest (e.g., alcohol, etc.).

In some examples, the sample capture medium can be formed from material that is selected based on the type of target substance or analyte. For example, for THC, the sample capture medium can be formed from hydrophilic felt material. Further, in some examples, the material can be woven and electrostatic. As an addition or variation, the sample capture medium can include, for example, cellulose, tissue paper, a paper towel, cotton, rayon, or other material that is hydrophilic but also has permeability so as to air flow therethrough. Still further, in examples, the material can be fibrous, e.g., textile-based (woven or unwoven), material that is hydrophilic in nature may be used. Such material may, in some implementations, have a thickness of 0.1 mm to 1 mm.

In embodiments, a sample collection device corresponds to a test cartridge that collects particles contained in a breath or ambient air sample. The collected particles can be captured on a membrane or medium during an air (or sample) intake action. Once a sample is sufficiently collected, the device can be manually manipulated to cause the sample to be immersed and sealed in a solution that stabilizes and maintains the sample. The device can then be transported to a laboratory or test facility, where the solution with the sample can be access and analyzed.

Further, in embodiments, a sample collection device is configured to be manipulated by one action, such as by an action where a user compresses or otherwise collapses one housing against another. The application of force by the user can be done manually or through use of machinery, such as a lever compression machine where a user pulls or manipulates a lever to exert a compression force on an item. The sample collection device can further be structured such that by performance of the action where the housing segments are manipulated in a designated manner (e.g., housing segments or compressed or otherwise collapsed against one another), the collected sample is immersed and sealed in a solution from which particles of the sample can subsequently be eluted and analyzed for a target substance (e.g., THC).

Examples include a sample collection device that includes a housing, a reservoir structure and a sample capture interface. The housing includes an inlet to receive an air sample from a subject. The sample capture interface provided with the housing includes a sample capture medium. The sample capture interface is manipulatable to insert the sample capture medium into the reservoir structure. Further, the sample capture interface seals the sample capture medium within the reservoir structure. Upon insertion of the sample capture interface, the sample capture interface seals the sample capture medium within the reservoir structure.

In examples, the sample capture interface can form the seal automatically, as a result of its insertion in the reservoir structure. The sample capture interface is structured to be form a second seal, for example, to retain the inserted sample capture medium and the preservation solution.

In examples, the air sample can be a breath sample, such as a breath sample collected from a subject or from ambient air conditions during a sample intake process.

Still further, in examples, a sample collection system includes a housing having an inlet to receive a sample. A sample collection interface is mateable within the housing to retain a sample capture medium. A reservoir structure is provided to retain the sample capture medium in a solvent or preservation solution.

In some examples, the sample collection system includes a pump device to fluidly couple with the housing to assist sample collection during a sample intake process. In some implementations, the reservoir structure is provided as a separate structure from the housing. In variations, the reservoir structure can be integrated into the housing. Still further, the housing may include a cap to seal an interior of the housing, including the reservoir structure. As an addition or variation, the sample capture interface includes an extension that enables the sample capture interface to be withdrawn and inserted into the housing.

In examples, sample collection device includes a top housing segment having an inlet to receive an air sample, a bottom housing segment, and a plunger structure provided with the top housing segment, where the plunger structure includes a shaft having a channel and an engagement end. The bottom housing segment includes a base structure having a reservoir structure, where the reservoir structure including a seal and a predetermined amount of preservation solution. Further, the sample capture medium is retained by the shaft at or near the engagement end. During a sample intake process, the device is configured so that an air sample is received through the inlet and guided through the sample capture medium. The top housing segment is manipulatable relative to the bottom housing segment to move from an extended position to a collapsed position, coinciding with the top and bottom housing segments being manipulated from an extend state into a collapsed state. Further, the movement of the housing segment relative to the bottom housing segment coincides with movement of the plunger structure, such that engagement end of the plunger structure is positioned and configured to pierce the seal of the reservoir structure, to submerge the sample capture medium in the preservation solution when the top housing segment is moved to the collapsed position.

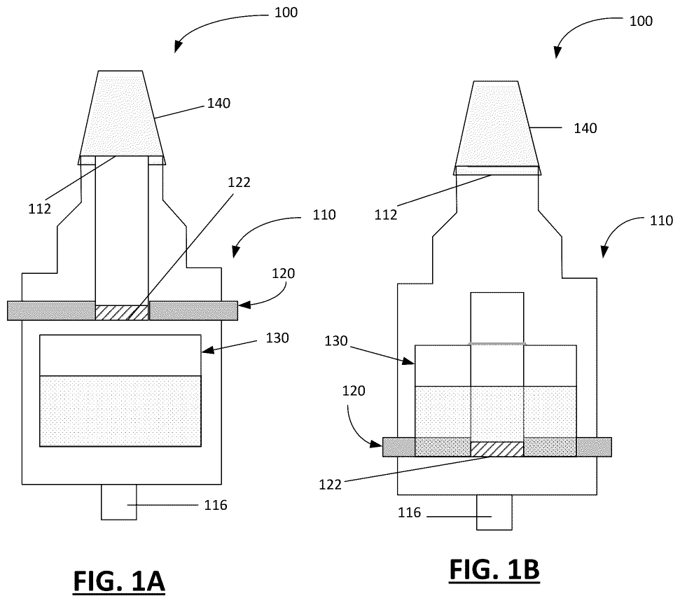

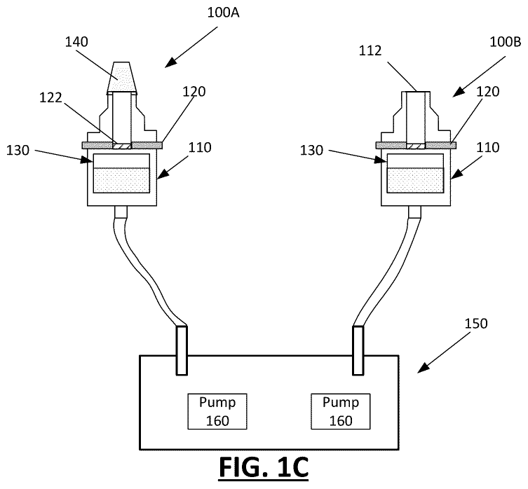

FIG. 1A through FIG. 1C illustrate a sample collection device and system, according to one or more embodiments. As described with various examples, a sample collection device 100 can be used to collect breath samples from a subject. The breath samples can be analyzed for target substances or analytes. With reference to FIG. 1A and FIG. 1B, a sample collection device 100 includes a housing 110, a sample capture interface 120, and a reservoir structure 130. The sample capture interface 120 can include a structure that retains a sample capture medium 122 in position to intersect a sample airflow received through an inlet 112 of the device 100. In examples, the sample airflow may correspond to exhaled breath provided by subject via a mouthpiece 140. In variations, the sample airflow can include air collected from a particular area, such as ambient air collected in the vicinity of a subject that is also providing a breath sample.

In some examples, the sample capture interface 120 can be manipulated relative to the housing 110 to immerse a sample in a preservation solution. In variations, the housing 110 can be manipulated to cause the sample capture interface 120 to immerse to sample in the preservation solution. In FIG. 1A, an operator can use the device 100 to implement a sample intake process to collect a sample. The sample intake process can include a subject breathing into a mouthpiece 140 that is attached to the device 100, such that the exhaled breath of the subject is received through the inlet 112. In some examples, the sample intake process for exhaled breath can include use of a pump (e.g., vacuum pump) that switches on when the user exhales. The device 100 and pump 160 can be coupled to one or more sensors (e.g., pressure sensors), such that the pump is controlled by detected conditions with respect to the sample intake process. The pump 160 can reside externally to the device 100, such as on a base station (see FIG. 1C). In such examples, the pump 160 can interface with the device 100 via tubing, through an air port or channel 116 that is formed on the housing 110. In examples, when a pressure sensor detects a subject exhaling into a mouthpiece 140, the pump may be triggered on, causing negative air pressure that facilitates the subject in breathing. When the user stops to take a breath, the pump 160 can stop to reduce intake of airflow that may contaminate the sample intake. When the user exhales again, the pump may be actuated to again facilitate the user. If conditions are detected where the subject is exhaling too hard, the condition can be detected and the pump can be switched off. In the case when the sample intake process collects ambient air, the pump can be operated for a duration of time to collect a sufficient amount of sample.

Once the sample intake process is complete, the operator can manipulate the housing 110 and/or the sample capture interface 120 to cause the collected sample to be immersed in the reservoir structure 130. In examples, the reservoir structure 130 is prefilled to include a preservation solution. In variations, the reservoir structure 130 can be filled with the desire preservation solution after the sample is received. The reservoir structure 130 can be in the form of, for example, a vial, made a material such as glass or plastic (e.g., polypropylene).

In some examples, an operator can perform a single action to insert a collected sample within the reservoir structure 130. For example, the sample capture interface 120 can be structured as a plunger that an operator can push inward, either through manipulation of the housing or (as shown) separately, to cause the collected sample to the inserted in the reservoir. In this way, the user can perform, for example, a single stroke to push the housing 110 and/or sample capture face 120 inward to immerse and seal the collected sample in a preservation solution.

Still further, in some examples, the device 100 can be structured or otherwise include features that allow for the sample to be automatically sealed within the reservoir. In examples, the sample capture interface 120 is structured, relative to the reservoir structure 130, to allow for one direction of travel. Once the sample capture interface 120 inserts the sample in the reservoir, a deformable layer provided with the sample capture interface 120 can seal the contents of the reservoir structure 130. Thus, the action performed by the user can immerse and seal the sample within the reservoir structure, while also locking the housing 110 in its state.

Further, once a sample capture interface 120 inserts a sample in the reservoir structure 130, the device 100 can be structured to automatically lock, such that the contents of the reservoir structure 130 cannot be accessed without causing destructive effect to the housing 110. In this way, the device 100 can be transported (e.g., shipped) to a site of a laboratory, where technician can inspect the device 100 for tampering, before accessing the sample with the solution from the reservoir structure 130.

As described with examples, the device 100 allows for an operator to collect and preserve a sample at a first site, then send the device with the sample preserved to a second site, where the sample can be analyzed for the presence of target substance(s). Thus, for example, an operator can collect a sample at a roadside, office, or other testing site. The sample collection device 100 can seal the sample in a sample preservation solution, such that the device 100 can be shipped (e.g., by mail or courier) to a laboratory or other side where the sample can be accessed and analyzed.

In examples, the preservation solution provided with the reservoir structure 130 elutes the collected sample from the sample capture medium. In some examples, the preservation solution includes methanol. As another example, the preservation solution includes glycerin.

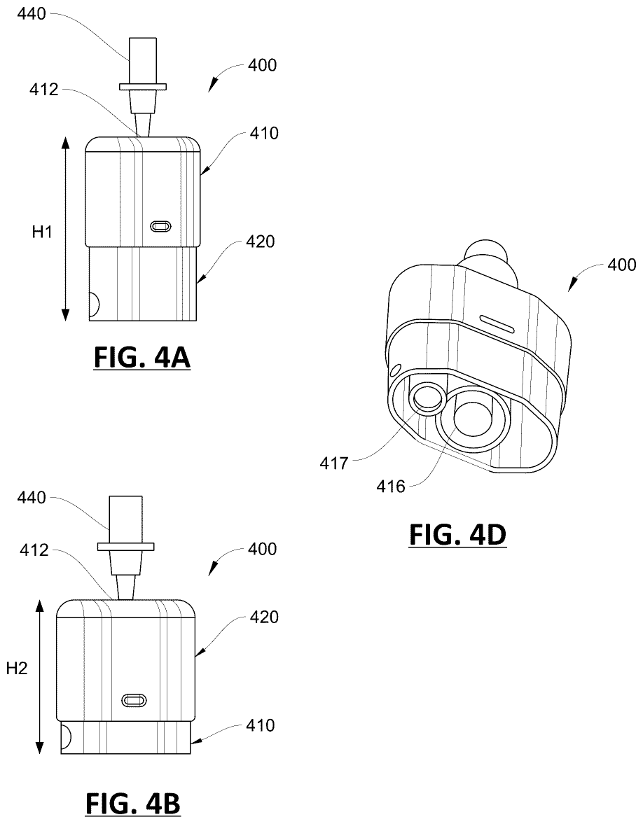

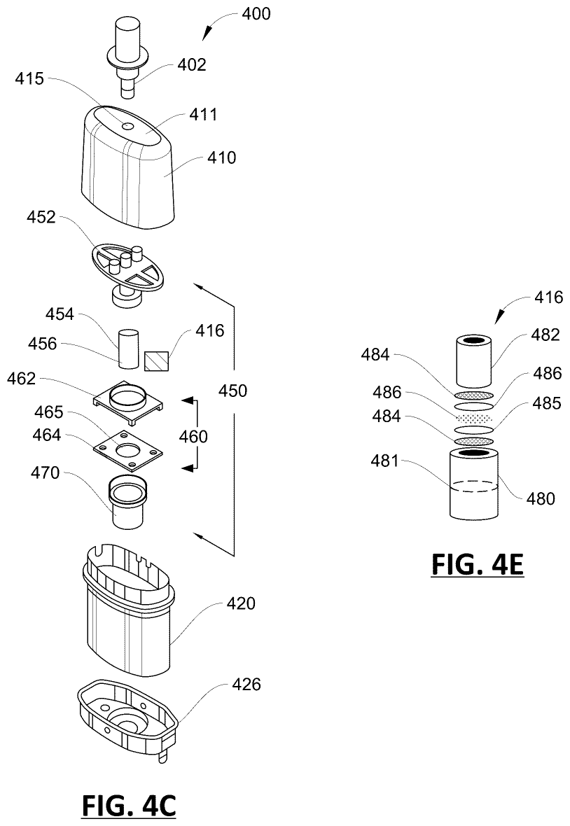

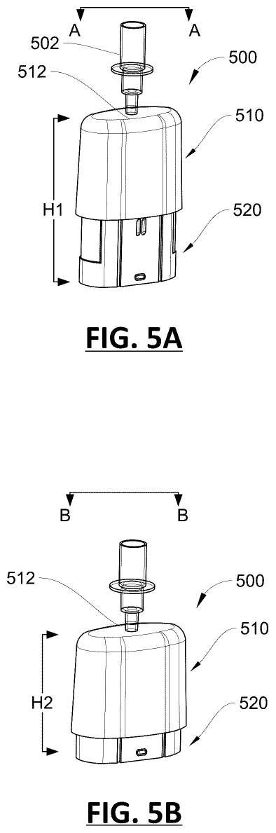

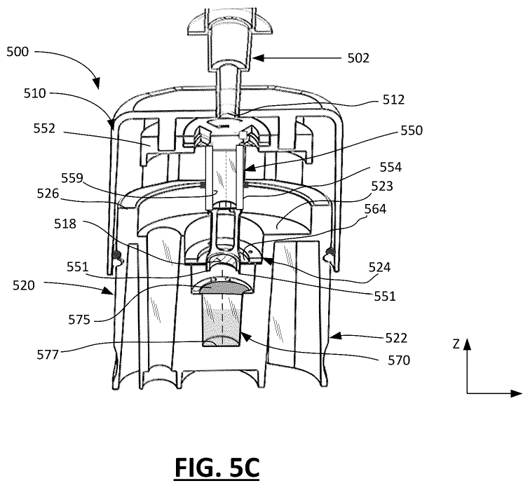

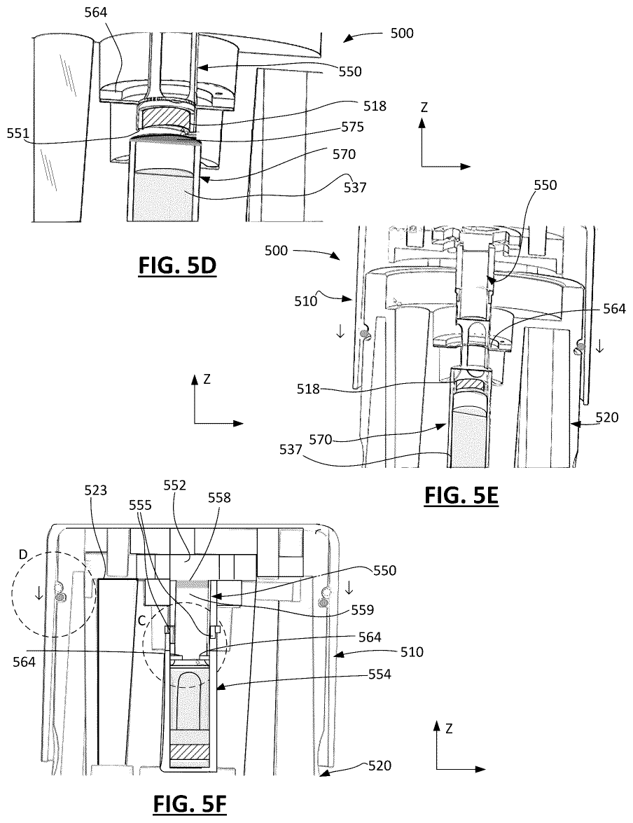

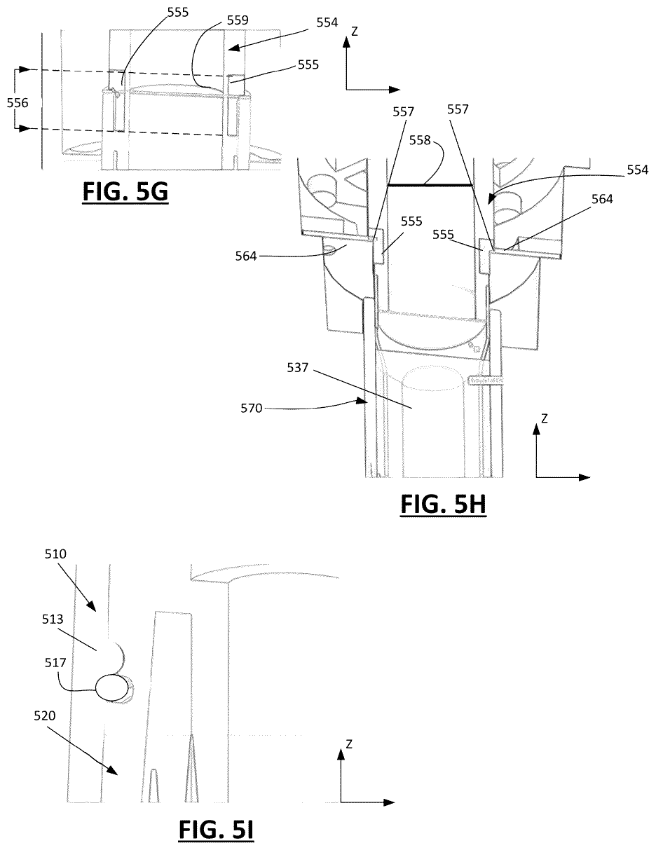

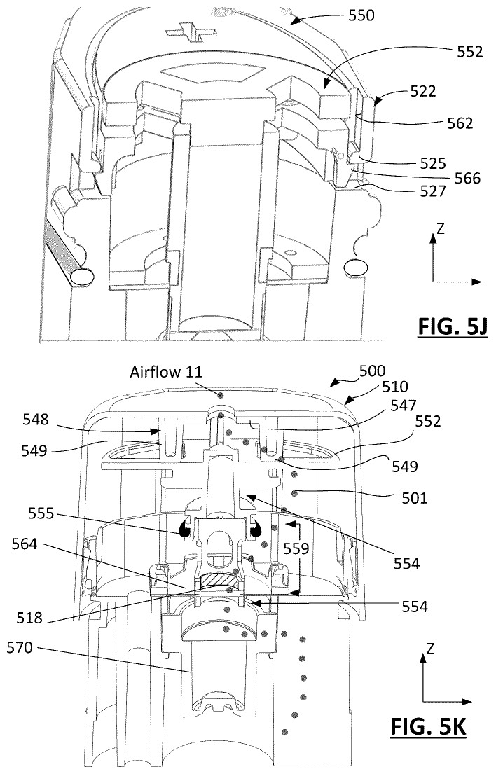

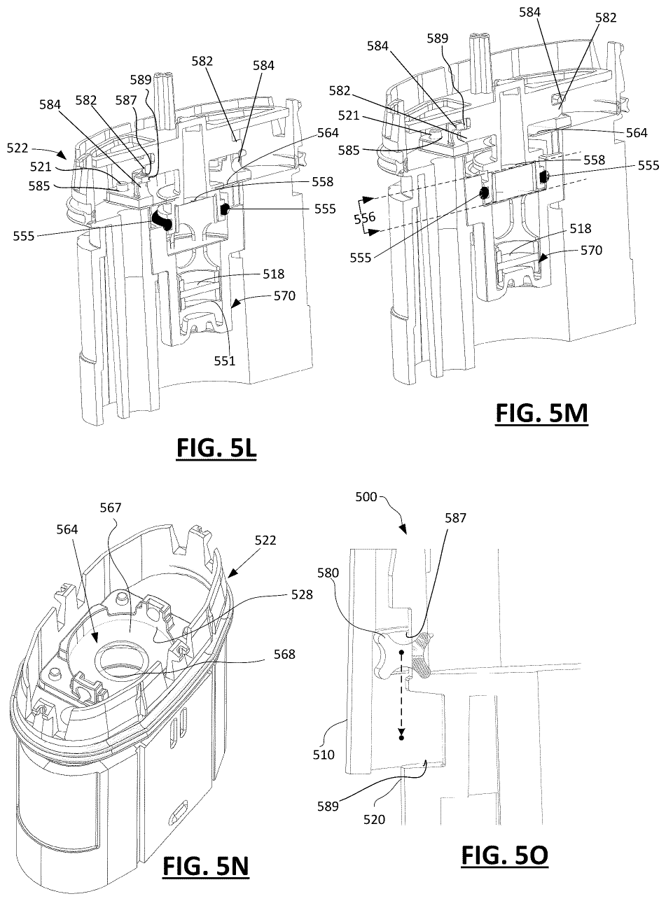

FIG. 1C illustrates a sample collection system, according to one or more embodiments. In examples, sample collection system includes a first sample collection device 100A, a second sample collection device 100B, and a base station 150. The device 100A can be constructed in accordance with embodiments such as described, and used to collect an exhaled breath sample from the subject. A subject can breathe into a mouthpiece 140 while the device 100A is fluidly connected to the base station 150. The base station 150 can be deployed with examples described herein, including examples of FIG. 4A through FIG. 4E, and FIG. 5A through FIG. 50. A pump 160 can operate within the base station 150 to draw air through the device 100A, in coordination with the subject exhaling. The sample capture medium 122 intersects the exhaled breath of the subject to capture particles contained in the subject's breath. Once a sufficient amount of sample is collected, depending on implementation, the sample collection interface 120 can be manipulated directly, or through the housing 110, to cause the sample capture medium 122 to move into the reservoir structure 130. The sample capture medium 122 can be immersed in a preservation solution that can be used to subsequently elute the sample particles for analysis.

In some examples, an ambient air sample is taken for each subject. The sample collection device 100B can be operated in combination with a pump 160 of the base station to collect ambient air that is within a designated vicinity of the subject who provided the breath sample. Each of the first sample collection device and the second sample collection device can be uses to implement a corresponding sample intake process under control of, for example, the base station (through the use of the pump) to concurrently collect a breath sample and an ambient air sample. The pump 160 can operate to draw air in through the inlet 112 of the device 100B. Particles contained in the ambient air can be collected in the sample capture medium 122. Once a sufficient amount of sample particles is captured, depending on implementation, an operator can manipulate the sample collection interface 120 directly or through the housing 110, to cause the sample capture medium 122 to move into the reservoir structure 130. In examples, two samples are captured and preserved in separate preservation solutions, where the first sample originates from the subject's breath, and the second sample originates from ambient air surrounding the subject. Depending on implementation, the sample collection devices can include optical markers, RFID tags and/or other identifiers that link the devices 100A, 100B to a particular subject. The devices 100A, 100B can then be transported (e.g., shipped by courier or process) to laboratory or other site for analysis. Subsequently, samples collected by each of the devices 100A, 100B can be analyzed, with the ambient air sample providing a baseline, point of reference or measure of secondary contamination with regards to the target substance or analyte. For example, the device 100B can be used to determine whether a detected target substance (e.g., THC) is attributable to secondary smore rather than direct inhalation by the subject.

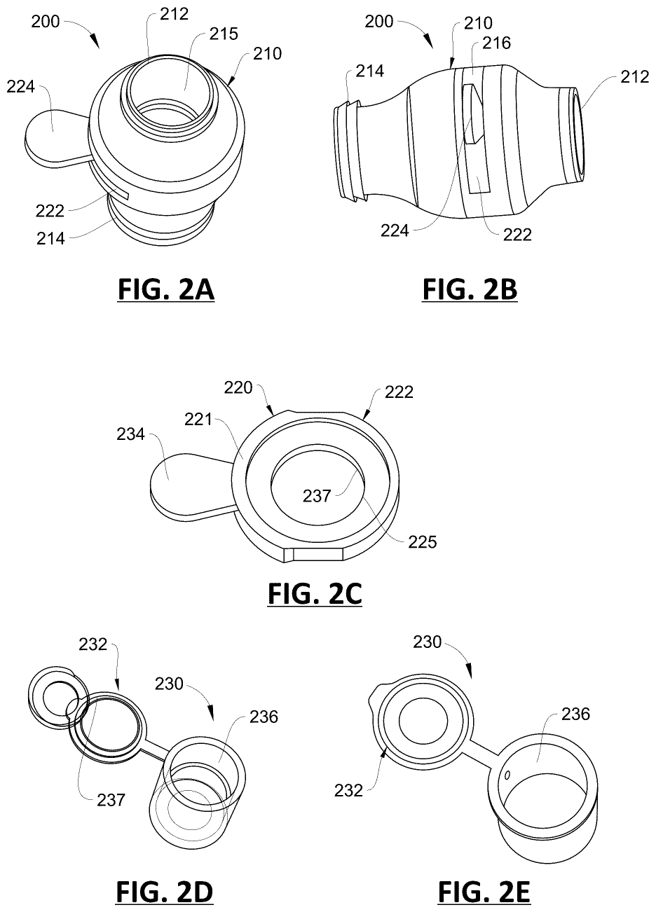

FIG. 2A through FIG. 2C illustrate a sample collection device 200, in accordance with one or more embodiments. FIG. 2A illustrates a top perspective view of the sample collection device 200. FIG. 2B illustrates a side view of the sample collection device 200. FIG. 2C illustrates a sample capture interface 220 for use with the sample collection device 200, according to one or more examples.

With reference to FIG. 2A and FIG. 2B, the device 200 includes a housing 210 including an inlet 212 and a bottom end 214. At least one channel 215 extends through the housing 210, extending from the inlet 212 to the bottom end 214. The housing 210 can also include a peripheral slot 216 to receive the sample capture interface 220. In use, the inlet 212 receives a sample, coinciding with the intake of airflow through the channel 215. Depending on implementation, the airflow coincides with a subject breathing through a mouthpiece that extends or connects to the inlet 212. As an addition or variation, the airflow coincides with a pump that draws air from a given region that is being sampled. In examples, the airflow can correspond to ambient air, such as may be collected in a given region or environment.

In examples, the bottom end 214 of the housing 210 can include an interface (e.g., tube, orifice, etc.) to a pump, such as a vacuum pump. In some variations, the pump can be operatively coupled to a sensor (e.g., pressure sensor) that can control when the pump is turned on and off to facilitate intake of sample collection. In some examples, the pump can be configured to switch on when, for example, the user breathes or performs a sample intake operation. For example, the pump can use a pressure sensor to detect the pressure level at the inlet 212. When the pressure rises (e.g., coinciding with the user exhaling), the pump can turn on to provide vacuum pressure via the bottom segment 214, to facilitate sample collection when the user exhales. When the user stops exhaling, the change in pressure can be detected by the sensor and the vacuum pump can stop, so that no sample collection is performed when the user stops breathing. In similar fashion, the vacuum pump can be switched on when ambient air is being collected as the sample. The vacuum pressure can be applied through the chamber 215 via the bottom segment 214 to collect the ambient air sample.

With reference to FIG. 2A through FIG. 2C, the sample capture interface 220 includes a sample collection structure 222 having an opening 225 over which a sample collection medium can be affixed. For example, a sample capture medium can be affixed to the sample collection structure 222, with a majority of the sample capture medium being aligned over the opening 225. The sample collection structure 222 and opening 225 can be dimensioned to retain the sample capture medium over the channel 215 of the housing 210 when the sample collection structure 222 is inserted into the peripheral slot 216 of the housing 210. In this way, the sample capture medium occupies an axial thickness channel 215, where the airflow coinciding with the sample intake is intercepted by the sample capture medium, such that particles contained in an airflow sample can be collected in the medium. Further, the sample collection structure 222 can include a sealing structure, such as a lip protrusion 221 or structure. The lip protrusion 221 can include or otherwise be combined with a gasket, O-ring or other deformable material to enable the sample collection structure 222 to seal when inserted into a suitably dimensioned slot.

In application, a breath capture medium (not shown in FIG. 2A through FIG. 2C) is positioned over the opening 225, and the sample capture interface 220 with the breath capture medium are inserted into the peripheral slot 216 of the housing 210. The sample capture interface 220 can also include an extension 224 that extends outward from the sample collection structure 222, to enable the sample capture interface 220 to be manually gripped. In this way, the extension 224 can be gripped to enable the user to insert the sample collection structure 222 into the peripheral slot 216 of the housing 210.

In use, the sample capture interface 220 with the breath capture medium can be inserted into the peripheral slot 216 of the housing 210. When inserted, the breath capture medium intersects the channel 215, such that the sample airflow is directed into the breath capture medium. Once the sample is collected, the sample capture interface 220 can be removed from the peripheral slot 216 and inserted into a separate reservoir structure 230 (see FIG. 2D and FIG. 2E). As described with other examples, the sample capture interface 220 with the breath capture medium, or alternatively, the breath capture medium by itself, can be sealed within a reservoir device 230 for physical transport. In examples, the reservoir device immerses the sample (contained with the breath capture medium) in a solvent such that the sample will be stable and non-degraded when transported. The solvent can include, for example, methanol or glycerin.

FIG. 2D and FIG. 2E illustrate a reservoir structure 230 for use with an embodiment of FIG. 2A through FIG. 2C. The reservoir structure 230 can include a reservoir 236 and a cap 232 that can be manipulated to fit over the reservoir 236. The reservoir structure 230 and/or cap 232 can include an O-ring or other kind of seal to seal the interior of the reservoir 236, where the sample capture medium and solvent can be contained. In some examples, the reservoir structure 230 includes a peripheral slot 237 to receive the sample collection structure 222. The sample collection structure 222 can be inserted in the slow, with the lip structure 221 and/or other sealing mechanisms providing a seal once the sample collection structure 222 is fully inserted. The reservoir 236 may be pre-filled with solvent (see FIG. 2E), and the reservoir structure 230 combined with the sample capture medium can be transported to a facility where the cap 232 can be removed, and the sample capture medium can be analyzed. In variations, the cap 232 can be opened or removed from the reservoir 236, and the user can place the sample capture medium from the sample capture interface 220 into the reservoir 236. Still further, in other variations, the user can open the cap to add solvent, then seal the cap 232 and ship the reservoir structure 230 with the sample capture medium to a facility.

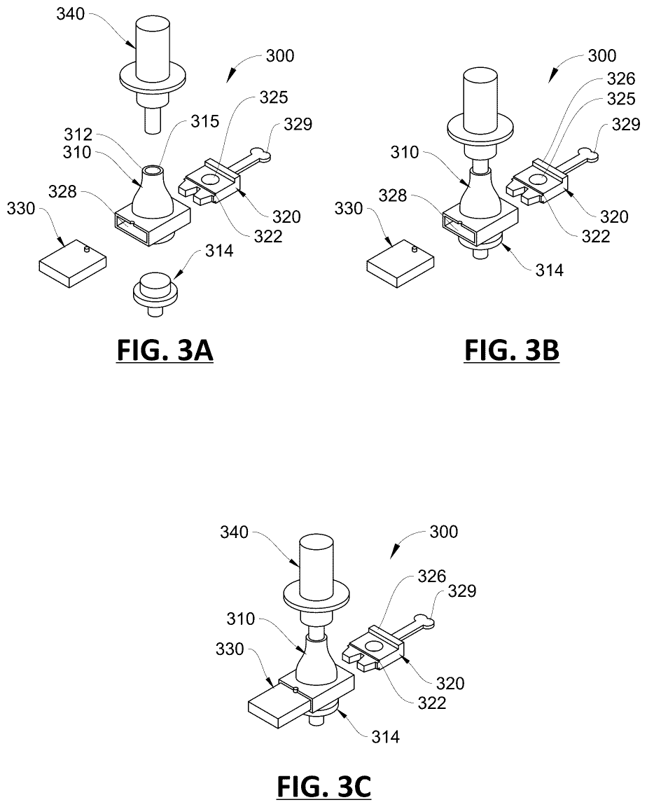

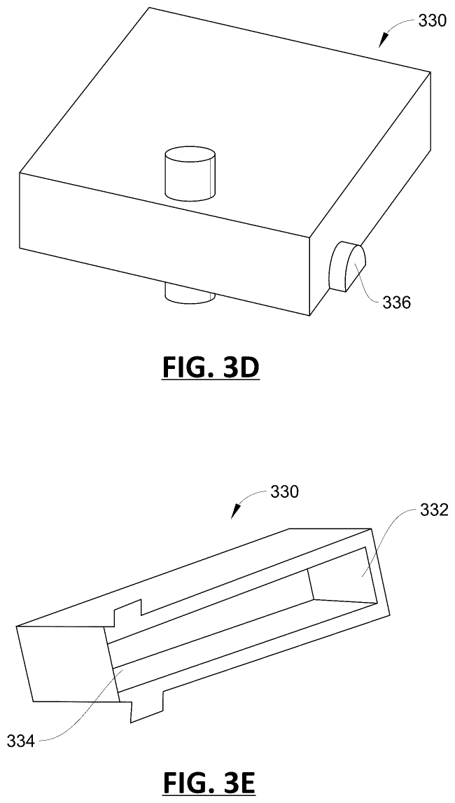

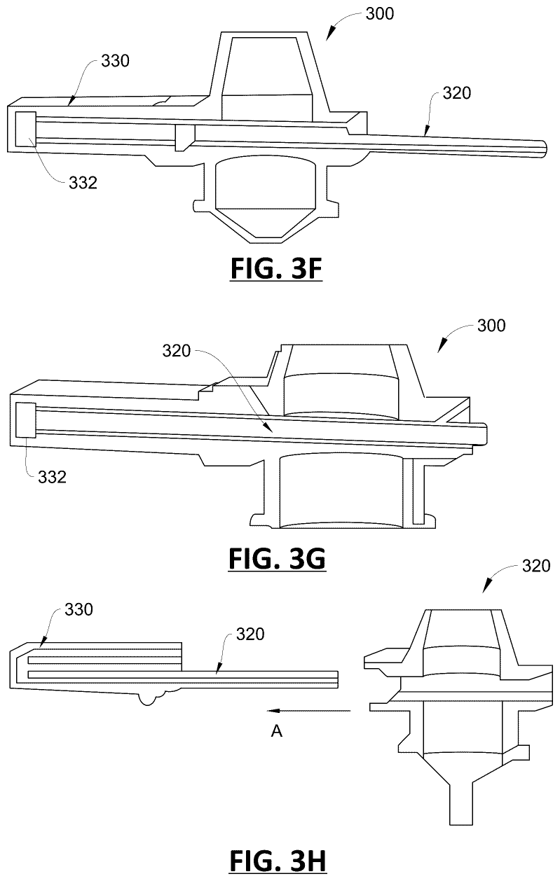

FIG. 3A through FIG. 3C provide an exploded view of a sample collection device 300, according to one or more examples. The sample collection device 300 includes a housing 310 having an inlet 312 and a bottom segment 314. The inlet 312 provides access to a channel 315 which extends axially through the housing 310. The housing 310 can include a peripheral slot to receive a sample collection interface 320. Additionally, the housing 310 includes a cartridge interface 328 to receive a cartridge 330. The bottom segment 314 can include one or more interfaces for mating the sample collection device 300 with a pump (e.g., vacuum pump). For example, a vacuum pump can be used to facilitate a sample intake process or action.

In examples, the inlet 312 is structured to receive a mouthpiece 340 or extension for facilitating intake of a sample. A sample intake process or action can include intaking air (e.g., breath, ambient air) through the channel 315 via the inlet 312. For example, a user can exhale into the mouthpiece 340 so that aerosol particles are forced through the channel 315.

The sample capture interface 320 can be structured to insert into a peripheral slot of the housing 310. As described with an example of FIG. 2C, the sample capture interface 320 can include a retention structure 322 to retain a sample capture medium 325, a sealing structure 326, and an extension 329. The sealing structure 326 can correspond to, for example, a lip structure and/or a layer of deformable material (e.g., rubber, elastomer, etc.) that surrounds a peripheral end of the retention structure 322. As described, the retention structure 322 can be dimensioned to snugly fit within the reservoir 332 of the cartridge 330, such that the sealing structure 326 can deform and seal when the retention structure 322 is inserted into the reservoir 332. The sample cartridge interface 320 is further configured to position a sample capture medium 325 across the channel 315 when the sample capture interface 320 is inserted into the housing 310. The sample capture interface 320 can be inserted prior to the sample intake operation or action is performed. When a sample is taken (e.g., user breaths into mouthpiece 340, pump captures ambient air, etc.), aerosol particles are captured on the sample capture medium 325.

With further reference to FIG. 3C, the sample collection device 300 can be assembled or partially assembled for sample capture on site. In examples, the cartridge interface 328 is mated with the cartridge 330. The cartridge 330 can be mated to the cartridge interface 328 so that the cartridge 330 is retained with the housing 310 when a sample intake process or action is performed.

FIG. 3D and FIG. 3E illustrates an example of cartridge 330, according to one or more examples. The cartridge 330 includes a reservoir 332 that can be filled with a solvent for maintaining a collected sample. In some examples, the reservoir 332 is pre-filled and sealed at an engagement end 334. For example, the cartridge 330 can be pre-filled with a preservation solution (e.g., methanol) and the engagement end 334 can be sealed with foil (not shown). The cartridge 330 can further include mating structures 336 which mate with the cartridge interface 328. In examples, an exterior side of the cartridge 330 includes external features 336 that friction or snap-fit with corresponding mating structures (e.g., openings) formed into the housing 310 of the sample collection device 300.

Prior to a sample being taken, the mouthpiece 340 can be mated to the housing 310 at the inlet 312. The cartridge 330 can be mated to the housing 310 at the cartridge interface 328 (e.g., the cartridge 330 can be snap fit into a corresponding mating structure of the cartridge interface 328). Further, the sample cartridge interface 320 can be inserted into the peripheral slot of the housing 310, where the user can use the extension 329 to manipulate the retention structure 322 into position. The sample can then be taken (e.g., user breathes into the mouthpiece 340), and aerosol particles are captured on the sample capture medium 325 of the sample cartridge interface 320. Once the sample capture medium 325 contains a sample, the sample cartridge interface 320 can be pushed through the slot of the housing 310 into and through the seal of the engagement end 334 of the cartridge 330.

FIG. 3F through FIG. 3H illustrate components of the sample collection device 300 being manipulated to immerse the sample captured medium (with the captured sample) in a solvent for transport to a remote site. In FIG. 3F, the sample collection device 300 is in a state where the sample capture interface 320 is inserted into the peripheral slot of the housing 310. In FIG. 3G, once the sample is collected, the sample cartridge interface 320 is pushed radially so that the retention structure 322 of the sample cartridge interface 320 (with the sample capture medium 325) is inserted into the reservoir 332 of the cartridge 330. The retention structure 322 pierces the seal at the engagement end 334 of the cartridge 330, such that the sample capture medium is immersed in a solvent within the reservoir 332. Further, the sealing structure 326 of the sample cartridge interface 320 can seal the retention structure 322 with the solvent within the reservoir 332 of the cartridge 330. In FIG. 3H, the cartridge 330 with the sample cartridge interface 320 is removed from the housing 310, as indicated by the directional arrow A. The cartridge 330 with the sample capture interface 320 sealed within the reservoir 332 can be then be transported or shipped to a remote site.

FIG. 4A through FIG. 4E illustrate another example of a sample capture device, according to one or more embodiments. A sample capture device 400 such as shown with examples of FIG. 4A through FIG. 4E can be used to implement a sample capture system such as described with FIG. 1C, or a sample capture device such as described with examples of FIG. 5A through FIG. 5O.

With reference to FIG. 4A and FIG. 4B, a sample capture device 400 includes a top housing segment 410 and a bottom housing segment 420. The top housing segment 410 includes an inlet 412 that can optionally receive a mouthpiece 402. As described with examples, the device 400 can be operated to receive a sample through airflow (e.g., subject exhaling breath, ambient air collection, etc.). When the sample is collected, the housing segments 410, 420 can be manipulated to cause the sample to be sealed within the device 400, so that the sample can be transported for analysis at a laboratory at another location.

In examples, the housing segments 410, 420 can be manipulated by a user collapsing or contracting the housing segments 410, 420. Prior to the sample being collected, the sample collection device 400 is constructed of a top housing segment 410 and a bottom housing segment 420, where the top housing segment 410 is manipulative relative to the bottom housing segment 420 to move from an initial extended or elongated position (coinciding with an initial or extended state) downward over a portion of the bottom housing segment 420, to lock or fix into a collapsed or contracted position (coinciding with a collapsed or contracted state). As show, a height H1 of the sample collection device 400 in the initial or extended position is greater than the height H2 of the sample collection device 400 in the collapsed or contracted state. While the sample is being collected, the housing segments 410, 420 are positioned in an initial or extended state (where the length is represented by H1, see FIG. 4A). Once the sample is collected, a user can press the housing segment 410 downward, causing the top and bottom housing segments 410, 420 to collapse towards one another, such that the top and bottom housing segments 410, 420 are in the collapsed (or contracted) state (where the length is represented by H2, and H1>H2). The act of collapsing the housing segments 410, 420 can include the top segment moving downward, with a periphery of the top housing segment moving over a portion of the bottom housing segment 420. Further, the act of collapsing the housing segments 410, 420 causes the collected sample to be immersed and sealed in a solution. According to examples, once the sample is collected, the user can perform a single action (e.g., press top housing segment 410 downward) to cause the sample to be packaged, or otherwise ready for shipment or transport. In variations, a user can operate a lever or machine to generate the compression force for the sample capture device 400.

When the sample intake process is complete, an operator can collapse the respective top housing segment 410 into the bottom housing segment 420, causing the collected sample to be submerged and sealed within a solvent (e.g., methanol) that preserves the sample. The sample collection device 400 can then be packaged and transported (e.g., shipped through a carrier) to a laboratory for processing of the collected sample, which is sealed and preserved within the sample collection device 400.

FIG. 4C illustrates an exploded view of the sample collection device 400, according to one or more examples. The sample collection device 400 includes top housing segment 410, plunger structure 450 structured to retain a sample capture medium 416, a diaphragm layer 460 and a reservoir structure 470. As described, the diaphragm layer 460 and sample capture medium 416 form a sample capture interface 424 for the collection of particles in a breath or air sample.

In greater detail, the top housing segment 410 includes an inlet 415 formed on a top surface 411. An optional mouthpiece 402 can be mated with the inlet 415. The mouthpiece 402 can include an opening to receive breath from a subject, a saliva trap, and an outlet that directs captured breath into the housing of the sample capture device 100. The plunger structure 450 can be integrated or otherwise coupled with one of the housing segments 410, 420, such that the act of collapsing the housing segments 410, 420 causes the plunger structure 450 to move in an axial direction (along axis Z) along the top shelf 452 and shaft 454.

The shaft 454 can provide a channel to retain a sample capture medium 416 that intersects the airflow of the sample. The sample capture medium 416 can be formed from, for example mesh material to capture particles in the breath/air sample. A diaphragm layer 460 can form a seal about the shaft 454, such that an air sample received through the inlet 415 during a sample intake process is directed through the sample collection medium 416, and out through an exit 426 (see FIG. 4D) of the device 400. In examples, the diaphragm layer 460 includes a retention structure 462 to secure a diaphragm 464 in position across a lateral thickness of the bottom housing segment 420. In examples, the diaphragm 464 can be formed from resilient material that is impervious to airflow. An opening 465 is formed in the diaphragm 464 to receive a segment of the shaft 454. The opening 465 can be dimensioned to allow axial movement by the shaft 454, while at the same time preventing air of the sample from flowing around and past the sample capture medium 416.

In examples, the sample collection device 400 may be connected to operate with a vacuum assist port to facilitate the drawing in of a breath or air sample, such as through the act of a user breathing through a mouthpiece 402. As further described, a sample capture medium (e.g., mesh material) can capture particles present in the breath/air sample during a sample intake process. To capture a breath sample, a subject may breath through, for example, the mouthpiece 402, under assist by a connected or integrated vacuum pump. One or more sensors (e.g., pressure sensors) can be integrated with the operation of the vacuum pump to trigger the pump to turn on at the moment the subject is breathing (e.g., coinciding with a rise in pressure above a threshold level from the subject breathing into the mouthpiece 402). When the user stops breathing, the drop in pressure can also be detected, so that the pump drops off. In the case where the sample is ambient air, the vacuum pump can switch on for a given interval until a sufficient amount of sample has been captured. For each type of sample, the air intake for the samples is directed through the breath capture medium where the particles are captured.

In examples, the reservoir structure 470 is retained fixed in position within the bottom housing segment 420, axially aligned with the shaft 454 and the diaphragm opening 465. The reservoir structure 470 can be pre-filled with a preservation solution such as methanol, of a quantity that is sufficient to fully immerse the sample capture medium 416 without the immersion causing spillage. The reservoir structure 470 can include a seal (e.g., foil) to retain the preservation solvent until use. A bottom engagement end 456 of the shaft 454 can be structured (sharpened, formed from metal, etc.) to break the seal of the reservoir structure 470 when the housing segments 410, 420 are collapsed.

When the housing segments 410, 420 are collapsed, the plunger structure 450 is moved axially relative to the bottom housing segment 420, such that a portion of the shaft 454 containing the sample capture medium 416 is moved into the reservoir structure 470 at sufficient depth, so as to be fully immersed in solvent (e.g., methanol). Further, the shaft 454 is structured to seal the reservoir structure 470 once the original seal is pierced and the shaft 454 is fully inserted into the reservoir structure 470. For example, a gasket or perimeter ring of deformable material can be provided to enable the shaft 454 to insert into the opening of the reservoir structure 470. Upon insertion, the material can deform to provide a seal about the perimeter of the shaft 454 near the top end of the reservoir structure. The interior of the shaft can also be structured to form a seal at a top end of the channel portion of the shaft, to prevent fluid from the reservoir structure 470 from escaping when the shaft 454 is fully inserted. Additionally, as described with examples, the top and bottom housing segments 410, 420 can include coupling structures (e.g., O-ring, X-ring) to inhibit movement of the housing segments from out of the collapsed position, thereby further enabling an effective seal of the reservoir structure 470 when the housing segments 410, 420 are collapsed. In this way, once sample collection is performed, the housing segments 410, 420 can be collapsed or otherwise manipulated to immerse the collected sample in a preservation solution. Further, as described in more detail, the manipulation of the housing can result in the housing segments 410, 420 being affixed to one another, with the sample being sealed within a preservation solution within the device 400.

FIG. 4D illustrates a bottom view of the sample collection device 400, according to one or more embodiments. As shown, the bottom housing segment 420 includes multiple air ports, including a forced (or vacuum) air port 417 and a sensor port 419. The air port 417 can connect (e.g., via tubing) with the pump, such as a vacuum pump, which operates to facilitate intake of airflow from which a sample can be acquired. In examples, the air port 417 connects to a vacuum pump provided with a base station that can operate near the device 400. The sensor port 419 can connect to a sensor, or set of sensors, where the pressure and/or other characteristics of air flow being received through the inlet 412 of the device 400 can be measured.

The intake of airflow can correspond to an exhaled breath, and aerosol particles contained in the exhaled breath are captured by the sample capture medium 416. As an addition or variation, the intake of airflow can correspond to air collected from a designated area. As described with an example of FIG. 1C, ambient air can be collected from a vicinity where an exhaled breath sample is taken, in order to measure or otherwise detect the presence of target particles (e.g., secondary smoke) in the vicinity of the subject providing the exhaled breath sample. In this way, some examples provide for the device 400 can have multiple uses, including a first use for collecting an exhaled breath sample of a subject, and a second use for collecting an ambient air sample.

While the sample intake process is performed, a vacuum is applied to the air port 417. For example, a tube (not shown) can connect the air port 417 to a base station, handheld or external system that includes a vacuum pump and/or airflow sensor. Alternatively, the device 400 can be integrated with a vacuum pump and/or airflow sensor.

In some examples, the sample capture medium 416 can be formed of, or otherwise include one or more layers of material that are selected to capture particles of a target type, where the layer(s) of the sample capture medium 416 are secured to an interior of the housing for the sample capture medium. The materials and structure of the sample capture medium 418 (including the number of layers) can be selected based on attributes or properties of the target particles (e.g., analytes). For example, the particle size for breath samples can vary between 0.05 μm-10.00 μm, and in some examples, 0.5 μm to 5.0 μm. In some applications, the materials can include hydrophilic felt material. Still further, as an addition or variation, the materials can be formed from material that are woven and electrostatic. Still further, the layers of the sample capture medium 416 can be formed from textile-based materials (e.g., woven), cellulose, tissue paper, a paper towel, cotton, rayon, or air-permeable and hydrophilic or hydrophobic material.

FIG. 4E illustrates an example of a sample capture interface, according to one or more embodiments. In some variations, the sample capture medium interface include one or more layers of select material that are housing within a containment structure. For example, the sample capture interface can include one or more layers of woven and electrostatic material. As an addition or variation, the one or more layers can be formed from textile-based materials (e.g., woven), cellulose, tissue paper, a paper towel, cotton, rayon, or air-permeable and hydrophilic material. In examples, the sample capture interface includes an outer shell 480 and an inner shell 482 that seats within a 481 ledge of the outer shell 482. One or more layers (collectively, the sample capture medium 416) are retained by the outer and inner shells 480, 482 respectively.

In variations, the layers of the sample capture medium 416 can include, for example, multiple layers, which include one or more mesh layers 484, one or more particle filters 486 and/or a bead layer 485 (e.g., silica bead layer). In some examples, the layers may be laminated and/or adhered to one another. For example, the layers may be stacked within the outer shell (e.g., on the shelf 481), and then the inner shell 482 is mounted onto the layers. In some examples, the mesh layer 484 can be formed from polytetrafluoroethylene (PTFE). The one or more layers of particle filters 486 can be formed from electrostatic filter media, such as manufactured under the trade name TECHNOSTAT PLUS (e.g., TECHNOSTAT 70 PLUS, TECHNOSTAT 90 PLUS, etc.). The bead layer 485 can be formed from, for example, silica.

FIG. 5A through FIG. 5O illustrates another example of a sample capture device, according to one or more embodiments. A sample capture device 500 such as shown with examples of FIG. 5A through FIG. 5O can be used to implement a sample capture system such as described with FIG. 1C. Further, a sample collection device 500 as shown by any of the examples of FIG. 5A through FIG. 5O can correspond to and/or operate in accordance with some or all of the features and functions of the sample collection device 400, as shown with examples of FIG. 4A through FIG. 4D.

With reference to FIG. 5A and FIG. 5B, a sample capture device 500 includes a top housing segment 510 and a bottom housing segment 520. The top housing segment 510 includes an inlet 512 that can receive an optional mouthpiece 502. As described with examples, the device 500 can be operated to receive a sample through airflow (e.g., subject exhaling breath, ambient air collection, etc.). When the sample is collected, the housing segments 510, 520 can be manipulated to cause the sample to be sealed within the device 500, so that the sample can be transported for analysis at a laboratory at another location.

In examples, the housing segments 510, 520 can be manipulated by a user collapsing or contracting the housing segments 510, 520. Prior to the sample being collected, and while the sample is being collected, the housing segments 510, 520 are positioned in an extended state (where the length is represented by H1). Once the sample is collected, a user can press the housing segment 510 downward, causing the top and bottom housing segments to collapse, such that the top and bottom housing segments 510, 520 are in the collapsed (or contracted) state (where the length is represented by H2, and H1>H2). The act of collapsing the housing segments 510, 520 causes the collected sample to be immersed and sealed in a solution. According to examples, once the sample is collected, the user can perform a single action (e.g., press top housing segment 510 downward) to cause the sample to be packaged, or otherwise ready for shipment or transport.

FIG. 5C illustrates a cross-sectional view of the sample capture device 500 along an axial axis Z (coinciding with lines A-A), showing the sample capture device 500 in an extended state, according to one or more embodiments. In FIG. 5C, the sample collection device 500 is shown in an extended state, coinciding with a time that precedes or is during the sample intake process. As shown, the mouthpiece 502 can mate with an inlet 512. A plunger structure 550 extends axially within the top housing segment 510. The plunger structure 550 can be connected to or integrated with the top housing segment 510 of the sample collection device 500. The plunger structure 550 can include a top shelf 552 that extends to a shaft 554. For example, the plunger structure 550 can be formed as a unitary or integrated component that is connected to or is otherwise integrated with the top housing segment 510.

In examples, the shaft 554 of the plunger structure 550 includes channel 559 that allows the passage of air along at least a portion of a length of the shaft 554. The channel 559 extends axially (along axis Z) to a sample capture interface 524. The sample capture interface 524 includes a sample capture medium (or portion thereof) 518, which can be retained at or just above a bottom engagement end 551 of the plunger structure 550. As described above with other examples, the sample capture medium 518 can correspond to a membrane or material that is pervious to air flow. Further, the sample capture interface 524 can include a diaphragm layer 564 and/or seal that forces the intake air flow to pass through the sample capture medium 518. In this way, the sample capture medium 518 can be positioned to intersect a sample airflow during a sample intake process, to capture particles (e.g., aerosol particles) of the air sample airflow (e.g., exhaled breath, ambient air, etc.).

In examples, the bottom housing segment 520 includes a base structure 522 that extends axially within the top housing segment 510. The base structure 522 includes a reservoir structure 570 that is positioned below the sample capture interface 524 and in alignment with the shaft 554 and channel 559 of the plunger structure 550. The reservoir structure 570 can be pre-filled under seal to retain a preservation solution for retaining a collected sample. Initially, the reservoir structure 570 can include a top seal 575 (see FIG. 5D) that seals the preservation solution within the confines of the reservoir structure 570. As described with other examples, the preservation solvent can include methanol, alcohol or glycerin.

As described, the top housing segment 510 can be moved in the axial direction (i.e., along the Z axis) towards the bottom housing segment 520 to cause the plunger structure 550 to move axially towards the sample capture interface 524. In variations, the plunger structure 550 can be moved independently or separately of the housing segments 510, 520. The operator can perform an action to collapse the housing segments 510, 520. The action causes a portion of the shaft 554 to pass through an opening of the diaphragm 564, and an engagement end 551 of the plunger structure 550 to pierce the seal 575 of the reservoir structure 570, followed by the sample capture medium 518 being moved axially into the confines of the reservoir structure 570, where it is immersed with the preservation solution. The engagement end 551 can be formed of metal and sharpened to pierce a foil that serves as the top seal 575 of the reservoir structure 570.

In examples, when the plunger structure 550 is fully inserted, the engagement end 551 can be positioned at or near a bottom 577 of the reservoir structure 570. When the plunger structure 550 is moved inward, the top shelf 552 can be received on an internal platform 523 of the base structure 522. The base structure 522 can also include a perimeter wall 526 that extends a height above the internal platform 523. The height of the perimeter wall can match or exceed a thickness of the top shelf 552, to form a stop on the axial movement of the top shelf 552 and thereby the plunger structure 550. In this way, the configuration of the base structure 522, as shown with the internal platform 523 can provide a stop for the axial movement of the shaft 554.

### Sample Insertion and Sealing

FIG. 5D through FIG. 5F illustrate mechanisms of the sample collection device 500 that combine to enable a collected sample to be inserted and sealed within the reservoir structure 570, in conjunction with the collapsing of the housing segments 510, 520, according to one or more embodiments. FIG. 5D illustrates a state just after when the collapse of the housing segments 510, 520 starts, when the sample capture medium 518 is pushed through the opening of the diaphragm layer 564 and before the sample capture medium 518 is submerged within the preservation solution 537. As the top housing segment 510 is moved towards the bottom housing segment 520, the engagement end 551 of the plunger structure 550 is moved towards a seal 575 of the reservoir structure 570, which may be prefilled with the preservation solution 537.

FIG. 5E illustrates a time during when the housing segments 510, 520 are being collapsed, when the engagement end 551 of the plunger structure 550 is moved axially to pierce the seal 575 of the reservoir structure 570. In this state, the sample capture medium 518 is positioned within the reservoir, but the plunger structure is not yet fully seated. As the housing segments 510, 520 are moved together, the plunger structure 550 travels to place the sample capture medium 518 at a desired depth within the reservoir structure 570, such that the sample capture medium 518 is immersed by the preservation solution 537 of the reservoir structure 570. The movement also pushes a length of the shaft 554 through the opening of the diaphragm layer 564. The shaft 554 of the plunger structure 550 is shown to be configured to maintain a seal on the reservoir structure 570 that prevents outflow from the reservoir structure 570, even as the shaft is entering the reservoir structure. The configurations include an outer deformable layer or gasket 555 and an internal cover 558 (e.g., foil) which combine to prevent the liquid of the reservoir structure 570 from leaking after or during when the insertion is taking place.

In FIG. 5F, the plunger structure 550 is shown fully inserted to immerse the sample capture medium 518 in the preservation solvent 537 of the reservoir structure 570. Further, in the collapsed state shown by FIG. 5F, the top shelf 552 of the plunger structure 550 is moved in position against the internal platform 523 of the bottom housing segment 520. Further, in examples, the contents of the reservoir structure are automatically sealed with insertion of the sample capture medium 518. The shaft 554 includes gasket 555, such as an O-ring that circumvents the perimeter of the shaft 554 at a select height, as well as an internal cover 558 which combine to prevent the contents of the reservoir structure 570 from leaking out.

FIG. 5G is a closeup of region C of FIG. 5F, illustrating an example mechanism for automatically sealing a sample capture medium within the reservoir structure 570. In examples, the shaft 554 includes a sealing segment 556 having a tapered length and a gasket 555. As the plunger structure 550 moves downward, a portion of the sealing segment 556 presses through the entrance of the reservoir structure 570, until the wider portion of the sealing segment 556 abuts the entrance of the reservoir structure 570, or a structure of the bottom housing segment 520 designed to receive the sealing segment 556. At the same time, the perimeter gasket 555 deforms and seals the shaft 554 in place relative to the reservoir structure 570. Further, the gasket 555 can be formed about the shaft 554, at a depth or position that allows for the desired length of travel for the sample capture medium 518 (e.g., to place the sample capture medium 518 on the bottom of the reservoir structure 570). When the plunger structure 550 is moved axially downward into the desired position (e.g., such that the sample capture medium 518 is at the bottom of the reservoir structure 570), the sealing segment 556 seals and affixes the shaft 554 in place against a rigid portion of the diaphragm layer 564. Alternatively, the sealing segment 556 affixes and seals the shaft 554 against an internal structure of the bottom housing segment 520. Still further, the gasket 555 can be sealed against the reservoir structure 570. In examples, the sealing segment 556 can be dimensioned in length or otherwise configured to enable variations to be provided with respect to, for example, the amount of solution contained in the reservoir structure 570, the relative position of the sample capture medium 518, and/or other design factors.

FIG. 5H illustrates a bottom perspective view of the region C, further illustrating a mechanism for sealing the reservoir structure 570 to prevent leakage after immersion of the sample capture medium 518. In examples, the shaft 554 can include a cover 558 (e.g., foil) or other type of seal that is provided across a cross-section of the shaft's hollow interior. When the shaft 554 is sealed in place, the cover 558 prevents fluid from the reservoir structure 570 from leaking through the hollow interior of the shaft 554. In examples, the shaft 554 can be structured to form a hermetic seal when the housing segments 510, 520 are manipulated to position the sample capture medium 518 within the reservoir structure 570. In this way, once the sample capture medium 518 is moved all the way into position, the housing segments 510, 520 are locked in place, and the shaft 554 of the plunger structure 550 is also sealed and affixed to the interior of the reservoir structure 570, with the cover 558 preventing the preservation solution from leaking from the reservoir structure 570. While an example of FIG. 5H illustrates the cover 558 above the gasket 555 after the housing segments are collapsed, in variations, the position of the cover 558 can be provided further below along the shaft 554, so as to be underneath the gasket 555 after the housing is collapsed.

In some examples, the gasket 555 can be formed from, for example, rubber, plastic or other deformable material. The material for the gasket 555 can also be selected for properties and attributes that include (i) non-reactiveness to the preservation fluid 537 (e.g., methanol) and sample, so as to not deteriorate or otherwise contaminate the sample by exposure to the contents of the reservoir structure 570; (ii) having sufficient durometer to withstand the compression and interaction with the structure that receives the sealing segment 556 when the housing segments 510, 520 are collapsed; and (iii) impervious to liquid and vapor. An example of a suitable material for forming the gasket 555 is VITON, manufactured by CHEMOURS of Delaware, United States. The shaft 554 can also include an internal cover (e.g., foil) to preclude fluid from escaping from the reservoir structure. In this way, the plunger structure 550 is able to move the sample capture medium 518 into the preservation solvent 537, and subsequently preserve the volume of fluid and the concentration of substances that form the sample. In some cases, the contents of the reservoir structure 570 can be maintained for days (e.g., three days), weeks or even longer, to allow time for the device 500 to be shipped or otherwise transported to a laboratory.

### Locking Mechanism

According to examples, the housing segments 510, 520 can be structured such that when the housing segments 510, 520 are moved into the collapsed position, the housing segments 510, 520 become locked or affixed to maintain the collapsed position. FIG. 5I is a closeup of region D of FIG. 5F, illustrating an example of the housing segments 510, 520 being structured to form a lock when manipulated into a collapsed position. The perimeter of the top and/or bottom housing segment 510, 520 can be shaped to have a draft angle that causes the fit between the respective segments to become tighter as the top segment 510 is moved over the bottom segment 520. A deformable layer 513 (e.g., O-ring, X-ring 580, see FIG. 5O) can be provided with the top housing segment 510 and positioned between the respective housing segments 510, 520 to implement a seal. When the top housing segment 510 is moved into the collapsed position, the deformable layer 513 can move (or snap down) into a divot 517 formed on the periphery of the bottom housing segment 520, thereby facilitating the creation of a seal that firmly maintains the housing segments 510, 520 in the collapsed position. In this way, the resulting seal prevents the housing segments 510, 520 from moving in either axial direction, such that the housing segments cannot separate or collapse further.

As an addition or variation, the housing segments 510, 520 can integrate other types of locking mechanisms that prevents tampering once a sample is acquired and preserved in a solution. FIG. 5J illustrates a partial perspective and cross-sectional view of the base structure 522 and the plunger structure 550 when the respective housing segments 510, 520 are in an expanded state (e.g., before or during sample collection).

With further reference to FIG. 5J, the top shelf 552 of the plunger structure 550 can be dimensioned to be separated from the base structure 522, so as to form a perimeter gap 562. In some examples, the top shelf 552 can provide a tang 566 that is maintained in the gap 562 when the housing segments 510, 520 are in the extended state (not shown in FIG. 5J). The base structure 522 can be shaped to provide a mating structure 525 for the tang 566. Once the housing segments 510, 520 are compressed, the top shelf 552 moves axially (or downward) and the tang 566 is flexed inward under bias when the mating structure 525 is encountered, thereby allowing for continued axial movement of the plunger structure 550.

As shown by FIG. 5J, once the plunger structure 550 moves inward the requisite depth (e.g., to place the sample capture medium 518 at the bottom of the reservoir structure 570), an opening 527 formed in the base structure 522 allows for the tang 566 to flex outward to an unbiased position. A portion of the tang 566 may then protrude from the opening 527. When the tang protrudes from the opening 527, the tang 566 securely resists forces that could otherwise separate the housing segments 510, 520. In this way, the tang 566 and mating structure 525 form a locking mechanism that prevents tampering with a collected sample. Further, separation of the housing segments 510, 520 would require removing the tang 566. As such, an attempt to access a collected sample would be readily detectable, as it would damage the tang 566 or device 500.

In examples, the device 500 can include housing structure to allow for an authorized person (e.g., laboratory where the device 500 is received) to access the sample in the reservoir structure 570. In an example, the top housing segment 510 can be separated to allow an authorized user to remove by force or through decoupling mechanism, a surface component that gives access to the reservoir structure via the hollow center of the shaft 554. For example, a technician can insert a pipette through the center of the shaft 554 to access the fluid within the reservoir structure 570.

### Sealing With Insertion Continued

FIG. 5K illustrates another cross-sectional view of sample collection device 500 when in the extended state, according to one or more embodiments. In an example of FIG. 5K, a support structure 548 is positioned between the underside of the top housing segment 510 and the plunger structure 550. The support structure 548 can provide support for the plunger structure 550 at the time that housing segments 510, 520 are collapsed. In particular, the support structure 548 can be configured to provide a direct force path for the plunger structure 550 when a force is applied to the outer surface of the top housing segment 510 to collapse the housing segments 510, 520. By promoting a direct force path, the support structure 548 better directs the plunger structure 550 to move axially, while minimizing lateral forces that would otherwise bend or impede the axial movement of the plunger structure 550, even when the applied force is off center or applied at an angle with respect to the axial axis Z.

In examples, the support structure 548 can be integrated, unitarily formed, or otherwise connected to at least one of the top housing segment 510 and/or the plunger structure 550. As shown, the support structure includes a top platform 547 that abuts or extends from the top housing segment 510. The support structure 548 includes a set of axial support members 549 that extends from the top platform 547 to the top shelf 552 of the plunger structure 550, to provide axial support. In an example shown, four axial support members 549 are provided. In variations, more or fewer axial support members 549 can be provided with the support structure 548. Further, the shape and size of the axial support members 549 can vary based on implementation. For example, the support structure 548 can include a single actual support member having a circular shape that interconnects or otherwise extends between the underside of the top housing segment 510 and the plunger structure 550.

Further, as shown, the shaft 554 of the plunger structure 550 can extend from the top shelf 552. When the housing segments 510, 520 are in the extended state, the sample capture medium 518 can be positioned over an opening of diaphragm layer 564 such that a sample received during a sample intake process is forced through the sample capture medium 518 before exiting the device via a vacuum or exit port (not shown). Further, as shown, the gasket 555 can be in the shape of an O-ring that surrounds the perimeter of the shaft. As described with other examples, the O-ring can be formed elastomeric material such as VITRON, manufactured by CHEMOURS.

FIG. 5K illustrates an airflow path 11 for a sample intake (e.g., breath or ambient air). The airflow 11 can be facilitated or driven by a vacuum pump that draws air in from a bottom of the device 500, via an exit or vacuum tube. The airflow 11 is drawn through the inlet 512, where it flows axially downward around an enclosed portion of the shaft 554 before flowing into the channel 559 and then through the sample capture medium 518. Except for the inlet 512, the interior of the device 500 is sealed, so that most of the drawn air during the sample intake process is passed through the sample capture medium 518. In some examples, a target volume of sample is taken using the device. For example, the sample intake process can be implemented such that a target volume of air is passed through the interior of the device 500 and through the sample capture medium 518. In this way, the concentration of target particles (e.g., THC) in a given amount of sample (e.g., X Liters, such as 10 L or 40 L) of human breath or ambient environment is determinable.

FIG. 5L and FIG. 5M are perspective cut-away views of FIG. 5K, showing the sample collection device with the top housing segment 510 removed while in a partially and fully collapsed state respectively, according to one or more embodiments. With reference to FIG. 5L, the partially collapsed state coincides with the plunger structure 550 positioning the sample capture medium 518 partially within the reservoir structure 570. The partially collapsed state can coincide with a moment in time when an operator applies force to the top housing segment 510 to collapse it. In this state, a portion of the shaft 554 has passed through the diaphragm layer 564, and the engagement end 551 of the plunger structure 550 has pierced the seal of the reservoir structure 570.

With reference to FIG. 5M, in the fully collapsed state, the sample capture medium 518 is fully submerged in the reservoir structure 570. The shaft 554 combines with the base structure 522 to form a sealing structure 556 to seal the contents of the reservoir structure 570. The sealing segment 556 provides for the gasket 555 to compress or deform against a receiving structure of the base structure 522. The internal cover 558 is positioned above the gasket 555 to prevent the contents of the reservoir structure 570 from escaping through the shaft 554.

In some examples, the housing segments 510, 520 can be structured to include one or more latching mechanisms that latch together when the housing segments 510, 520 are collapsed. With further reference to FIG. 5L and FIG. 5M, for example, each latching mechanism can be formed by a latch member 582 that is positioned and structured to engage a latch retainer 584 as the housing segments 510, 520 are collapsed. In FIG. 5L (before plunger structure 550 is fully inserted), the latch member 582 is shown aligned over the latch retainer 584 as the housing segments 510, 520 are moved towards one another. The latch member 582 can include an acutely angled surface 587 that slides against a counterpart surface 589 of the latch retainer 584, where the counterpart surface 583 is also slanted and angled to receive the latch member 582. The downward force to collapse the housing segments 510, 520 can be sufficient to compress or move one of the latch member 582 or latch retention structure 584 aside so that the latch member 582 passes below the latch retention structure 584 or latch receptacle (see FIG. 5M). At that point, the latch retention structure 584 is latched, meaning movement by the latch member 582 in the reverse direction (i.e., to separate the housing segments 510, 520) is precluded. One or multiple such latch mechanisms may be provided to latch the housing segments 510, 520 in place once the housing segments 510, 520 are latched. In this way, once the housing segments 510, 520 are collapsed, the housing segments remain collapsed (and latched).

As further shown by FIG. 5L and FIG. 5M, the base structure 522 of the bottom segment 520 can be affixed to the latch retention structure 584. For example, the base structure 522 can include a protrusion 521 that engages a corresponding opening of the latch retention structure 584. During assembly, the base structure 522 and retention structure 584 can be mated and assembled by aligning and fitting each protrusion 521 of the base structure 522 through an opening 585 of the retention structure 584. Once assembled, the protrusion 521 can be heated to seal the retention structure 584 against the base structure 522. Alternatively, the retention structure 584 can be affixed by adhesives. Still further, the latch retention structure 584 can be molded or unitarily formed as part of the base structure 522. By permanently affixing the retention structure 584 with the base structure 522, the plunger structure 550 cannot be separated from the base structure 522 without damaging the sample capture device 500 as a whole, in a manner that is readily detectable. In this way, the housing segments 510 can be structured to be tampered-proof, to prevent, for example, a user or third-party from accessing the reservoir structure 570 after the sample is taken.

### Base Structure and Diaphragm Layer

FIG. 5N illustrates an example of the base structure 522, according to one or more embodiments. The base structure 522 can include a receptacle 528 configured to retain the shaft 554 of the plunger structure 550 in the extended and collapsed states. The base structure 522 can include the diaphragm layer 564, which forms a circular opening to receive the shaft 554. In examples, the diaphragm layer 564 includes multiple layers formed from different materials and/or different thicknesses.

In an example of FIG. 5N, the diaphragm layer 564 includes two layers—a first layer extends from the base structure 522 and is formed from a first polymer layer 567 and a second polymer layer 568, where the two layers are compressed or adhered to one another. For example, the first and second polymer layers 567, 568 are laminated together. The second polymer layer 568 can extend annularly inward beyond the first polymer layer 567 to define an opening 565 for the diaphragm layer 564 where the shaft 554 can be received. The first polymer layer 567 can be characterized as having greater strength and less flexibility than that of the second polymer layer 568. In some examples, the first polymer layer 567 is formed from hardened plastic or rubber (or synthetic rubber), and the second polymer layer 568 is formed from thin film polymer. In an example, the first polymer layer 567 is formed from fluorinated ethylene propylene (“FEP”), and the second polymer layer 568 is formed from a material such as ethylene propylene diene monomer (“EPDM”) or neoprene.

FIG. 5O illustrates an example of sample collection device 500 that utilizes an X-ring 580 for a deformable layer. The X-ring 580 can be provided in place of, for example, an O-ring such as described with other examples. The X-ring 580 forms a barrier and seal between the top housing segment 510 and bottom housing segment 520. When the sample intake process is taking place, the X-ring 580 prevents air from escaping, thereby preserving the integrity of the sample. When compressed, the structure of the X-ring 580 enables relatively less compression (as compared to a comparable O-ring) to collapse the housing segments 510, 520. Further, the X-ring 580 enables a breakaway action where the X-ring and/or the top housing segment 510 can move axially once a compression force above a characteristic threshold is reached. A material as provided with the X-ring 580 can provide resistance up until a threshold force, at which point the structure of the X-ring folds and enables subsequent movement.

In example, the X-ring 580 can be positioned along a recess or divot 587 when the housing segments 510, 520 are in an extended state. Once collapsed, the X-ring can be moved into the divot 589.

### Flow Path Illustration

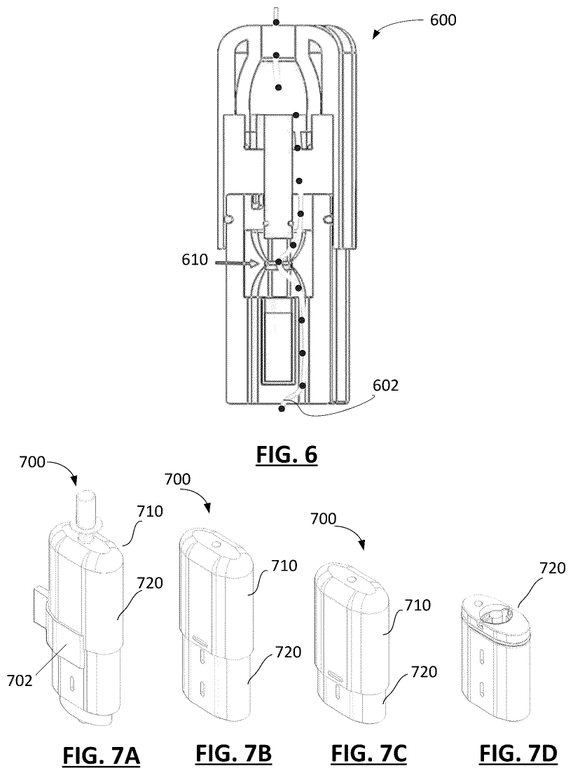

FIG. 6 illustrates a flow path for a sample intake of a sample capture device, according to one or more examples. A sample capture device 600 can be structured in accordance with one or more embodiments as described, including such as described with FIG. 4A through FIG. 4D and FIG. 5A through FIG. 5O. A flow path 605 resulting from a sample intake (e.g., user exhaling breath, collecting ambient air, etc.) where the airflow passes through a sample capture medium 610, before being guided through a bottom end of the device 600. As described with other examples, a pump (e.g., vacuum pump) can be fluidly connected (e.g., using tubing) to the bottom end 602 to facilitate or implement the sample intake. As further described, the sample intake is forced through the sample capture medium, prior to its being submerged in a preservation solution.

### Usage

FIG. 7A through FIG. 7D illustrate use of a sample capture device, in accordance with embodiments as described. In FIG. 7A, an unused sample capture device 700 can be operated at a first site to obtain a sample from a subject. The sample capture device 700 can include an external locking mechanism 702 that has to be forcibly removed or broken off, in a manner that prevents the mechanism from being reattached. The presence of the locking mechanism 702 can signify that the sample capture medium within the device is unused and therefore not contaminated.

In FIG. 7B, the locking mechanism 702 is removed by an operator (e.g., person administrating the sample intake process). Prior to or during the sample intake process, the housing segments 710, 720 are in an extended state.

In FIG. 7C, once a sample is acquired, the housing segments 710, 720 can be collapsed or otherwise manipulated, such that the device 700 is mechanically locked and the sample is inaccessible without damaging the device 700.

In FIG. 7D, the device 700 can be transported (e.g., shipped by courier) to a laboratory or other site where testing can be performed. A technician can remove the top housing segment 710 and manipulate locking mechanisms within the bottom housing segment 720 to access the solution containing the sample. For example, a lab technician can insert a pipette through a hollow shaft of a plunger mechanism to access the sample in a reservoir.

### Sample Intake

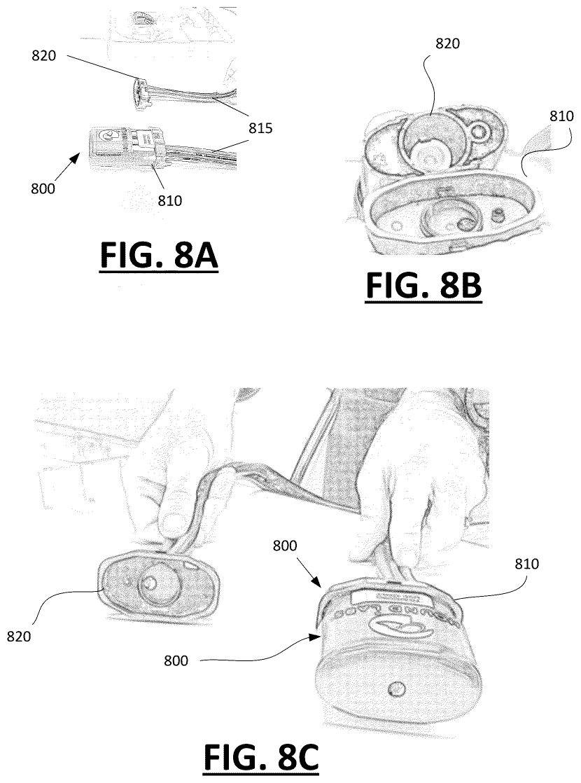

FIG. 8A through FIG. 8C illustrates a sample collection device 800 during a sample intake process, according to one or more embodiments. A sample collection device 800 can be configured or otherwise structured in accordance with embodiments described herein, such as, for example, sample collection device 400 as described with examples of FIG. 4A through FIG. 4D, and sample collection device 500 as described with various examples of FIG. 5A through FIG. 5O.

With reference to FIG. 8A through FIG. 8C, a bottom end 802 of the device 800 includes an interface 810 that extends to a flexible tubing 815 and to a second interface 820 for mating to a station that provides a vacuum pull. Each of the interface 810, 820 can include deformable material (e.g., polymer, rubber, foam). Further, the interface 810 can be structured to mate with the bottom end 802 of the device 800. Likewise, the interface 820 can be structured to mate with an outlet of a base station (or vacuum device). In examples, the interfaces 810, 820 is configured to securely retain the device 800. The deformable material contained within the irrespective interfaces 810, 820 can provide a seal to guard against leak of the breath/air sample during the sample intake process. Further, the deformable material can be selected to include thickness (e.g., foam) that allows for movement by the subject. The flexible tube 815 can also be of an adequate length to protect against movement by the subject causing a leak at the site of the adapter 810.

Although examples are described in detail herein with reference to the accompanying drawings, it is to be understood that the concepts are not limited to those precise examples. Accordingly, it is intended that the scope of the concepts be defined by the following claims and their equivalents. Furthermore, it is contemplated that a particular feature described either individually or as part of an example can be combined with other individually described features, or parts of other examples, even if the other features and examples make no mentioned of the particular feature. Thus, the absence of describing combinations should not preclude having rights to such combinations.

What is claimed is:

1. A sample collection device comprising:
a top housing segment having an inlet to receive an air sample;
a plunger structure provided with the top housing segment, the plunger structure including a shaft having a channel and an engagement end;
a bottom housing segment, the bottom housing segment including a base structure having a reservoir structure, the reservoir structure including a seal and a predetermined amount of preservation solution;
a sample capture medium retained by the shaft at or near the engagement end;
wherein during a sample intake process, an air sample is received through the inlet and guided through the sample capture medium;
wherein the top housing segment is manipulatable relative to the bottom housing segment to move from an extended position to a collapsed position, coinciding with the top and bottom housing segments being manipulated from an extend state into a collapsed state; and
wherein the movement of the housing segment relative to the bottom housing segment coincides with movement of the plunger structure, and wherein the engagement end of the plunger structure is positioned and configured to pierce the seal of the reservoir structure and to submerge the sample capture medium in the preservation solution when the top housing segment is moved to the collapsed position.

2. The sample collection device of claim 1, further comprising:
a sealing structure that is configured to form a seal for the contents of the reservoir structure by positioning of the sample capture medium within the preservation solution of the reservoir structure.

3. The sample collection device of claim 2, wherein the sealing structure includes a gasket that seals an airgap about the shaft when the top housing segment is moved to the collapsed position.

4. The sample collection device of claim 3, wherein the shaft includes an internal foil or cover to further seal the contents of the reservoir structure after the top housing segment is moved to the collapsed position.

5. The sample collection device of claim 4, where the gasket is an O-ring provided about the shaft.

6. The sample collection device of claim 4, wherein the gasket is formed from a polymer material characterized by being non-reactive to the preservation solution, and impervious to liquid and vapor.

7. The sample collection device of claim 6, wherein the sealing structure is configured to maintain a volume and an analyte concentration of the air sample for a period of at least 3 days.

8. The sample collection device of claim 1, further comprising a diaphragm layer that is positioned below or with the sample capture medium, to prevent airflow from exiting the sample collection device without first passing through the sample capture medium.

9. The sample collection device of claim 5, wherein the diaphragm layer includes multiple layers of elastic material.

10. The sample collection device of claim 1, further comprising a layer of deformable material provided between the top housing segment and the bottom housing segment.

11. The sample collection device of claim 10, wherein the layer of deformable material includes an X-ring.

12. The sample collection device of claim 1, further comprising a mouthpiece to mate with an inlet of the top housing segment, the sample intake process collecting a breath sample.

13. The sample collection device of claim 12, further comprising:
an air port to receive a tubing from a vacuum to facilitate the sample intake process.

14. The sample collection device of claim 13, wherein the sample intake process collects an air sample.

15. The sample collection device of claim 1, wherein the sample capture medium includes one or more of a woven material, an electrostatic material, a filter, a bead layer, and/or a layer of hydrophilic material.

16. A sample capture system comprising:
a first sample collection device;
a base station coupleable to the first sample collection device, the base station including a vacuum pump that is mateable to a air port of the first sample collection device, the base station being operable to control a sample intake process of the first sample collection device through use of the vacuum pump a top housing segment having an inlet to receive an air sample; and
wherein the first sample collection device is configured to captures a first sample from the first sample intake process, and to preserve the captured first sample in a preservation solution under seal.

17. The sample capture system of claim 16, wherein the first sample collection device include a sealing structure that is configured to preserve a volume and concentration of the collected first sample for a period of at least 3 days.

18. The sample collection system of claim 16, wherein the first sample collection device includes:
a plunger structure provided with the top housing segment, the plunger structure including a shaft having a channel and an engagement end;
a bottom housing segment, the bottom housing segment including a base structure having a reservoir structure, the reservoir structure including a seal and a predetermined amount of preservation solution;
a sample capture medium retained by the shaft at or near the engagement end;
wherein during a sample intake process, an air sample is received through the inlet and guided through the sample capture medium;
wherein the top housing segment is manipulatable relative to the bottom housing segment to move from an extended position to a collapsed position, coinciding with the top and bottom housing segments being manipulated from an extend state into a collapsed state; and
wherein the movement of the housing segment relative to the bottom housing segment coincides with movement of the plunger structure, and wherein the engagement end of the plunger structure is positioned and configured to pierce the seal of the reservoir structure and to submerge the sample capture medium in the preservation solution when the top housing segment is moved to the collapsed position.

19. The sample collection system of claim 16, further comprising a second sample collection device, the second sample collection device being coupleable to the base station concurrently with the first sample collection device, the second sample collection device being operable to implement a second sample intake process to collect a second air sample under control of the base station using the vacuum pump.

20. The sample collection system of claim 18, wherein the first sample intake process collects a breath sample, and the second sample intake process collects an ambient air sample.
